(19) United States
(12) Patent Application Publication
(10) Pub. No.: US 2024/0269460 A1
(43) Pub. Date: Aug. 15, 2024

(54) NEURAL INTERFACE WITH EDGE PROTECTED POROUS MATERIAL

(71) Applicant: INBRAIN NEUROELECTRONICS SL, Barcelona (ES)

(72) Inventor: José Antonio GARRIDO ARIZA, Sant Just Desvern, Barcelona (ES)

(21) Appl. No.: 18/567,731

(22) PCT Filed: Nov. 24, 2021
(86) PCT No.: PCT/EP2021/082726

§ 371 Date: Dec. 6, 2023

(30) Foreign Application Priority Data
Jun. 11, 2021 (EP) 21382525.0

(51) Int. Cl. A61N 1/05

(52) U.S. Cl. CPC A61N 1/0529

(57) ABSTRACT

The present invention relates to a neural interface device comprising an electrically conductive material, a porous material in electrical contact with the electrically conductive material, and a securing material for securing the porous material on the electrically conductive material, wherein the securing material comprises a protecting portion which is in physical contact with an edge region of the porous material.

The present invention provides a neural interface device, preferably a neural interface device for neurostimulation, e.g. for cortical and/or deep brain stimulation, a method for producing a neural interface device and a specific use of a polymer in this regard.

The present invention relates to the technical field of neural interfacing such as neurostimulation, in particular to cortical and/or deep brain stimulation. Cortical and/or deep brain Stimulation are methods for the diagnosis and therapy of neurodegenerative diseases such as inter alia the Parkinson's disease, epilepsy, and chronic pain. Electrical stimulation by means of leads which are implanted into brain areas or regions like the subthalamic nucleus and/or the globus pallidus internus can alleviate symptoms, such as tremor symptoms of a patient suffering from a drug-resistant Parkinson's disease. Further, the signals from a brain region or area at which the leads were implanted can be recorded and the state of the brain tissue can be determined using impedance measurements.

In this relation, neural interface devices are powerful tools to monitor, prevent and treat neural diseases, disorders and conditions by interfacing electrically with the nervous system. They are capable of recording and stimulating electrically neural activity once implanted in the nervous tissue. Currently, most neural interface devices apply electrodes interfacing with neural tissue. However, the term neural interface devices as used herein is not limited to electrode devices. Other kinds of neural interface devices are transistor devices.

Standard commercially available neural interface devices are based on metallic microelectrodes made of platinum Pt, platinum-iridium (Pt/lr), iridium oxide (IrOx) or titanium nitride (TiN). Those materials interact with the living tissue through a combination of Faradaic and capacitive currents, offer a limited chemical stability and are rigid. Metals' performance strongly drops in microelectrodes of tens of micrometres in diameter. Further, metals degrade over continuous tissue stimulation.

U.S. Pat. No. 7,813,796 describes an implantable electronic device comprising a hermetic electronics control unit, that is typically mounted on a substrate, that is bonded to a flexible circuit by an electroplated platinum or gold rivet-shaped connection. The resulting electronics assembly is said to be biocompatible and long-lived when implanted in living tissue, such as in an eye or car. The substrate can be a ceramic substrate, such as an alumina or silicon substrate.

In order to avoid some of the disadvantages associated with the above-mentioned metallic microelectrodes, electrodes with a coating applied to an electrically conductive material were suggested. However, such coatings bring along other disadvantages which include chemical and mechanical degradation of the coatings when the electrode is in operation.

WO 2010/057095 A2 provides an improved method for manufacturing an implantable electronic device. This publication describes a method of manufacturing an implantable electronic device, comprising the steps of providing a silicon wafer; building a plurality of layers including an electrode layer coupled to the wafer; coating the plurality of layers with an encapsulation; and modifying the encapsulation and at least one of the plurality of layers to expose an electrode site in the electrode layer. It further describes that a series of layers including a chromium layer and a gold layer are built and that a portion of the chromium layer is removed through an etching process to expose a portion of the gold layer. Vapor deposition is mentioned as a method for forming the series of layers. Nothing suggests that any of the gold or chromium layers might be porous.

With regard to stability against chemical and mechanical degradation of coatings, carbon based and in particular graphene based (coating) materials are very promising. This is not only because these materials are highly inert and mechanically robust and therefore avoid degradation problems. Graphene based materials are highly favorable also because of their electrical properties and flexibility. Graphene based materials thus appear ideal for safe electrical interfacing in aqueous environments.

Nevertheless, also for these carbon (coating) materials, further improvement is desired. Researchers are aiming at an even higher structural integrity. In particular, carbon material anchoring on the surface of the electrically conductive material should be so strong that even an initiation of carbon material delamination is avoided completely.

This is getting more and more important as electrode devices for neural interfacing are getting smaller and smaller. Miniaturized electrodes tend to have longer edges per area of tissue exposed active electrode surface and coatings in these areas therefore tend to delaminate more easily. Miniaturization has thus made safe anchoring of tissue exposed coatings on neural interface devices even more important than it was before.

It is thus an object of the present invention to enhance a device of the aforesaid art, and in particular to provide a neural interface device that meets highest requirements with regard to its structural integrity, even when equipped with very small or even brittle coated tissue exposed active areas.

The aforesaid object is achieved by the subject-matter of claim 1.

According to the present invention, a neural interface device is provided, comprising an electrically conductive material for transmitting electrical signals, a porous material in electrical contact with the electrically conductive material, and a securing material for securing the porous material on the electrically conductive material, wherein the securing material comprises a protecting portion which is in physical contact with an edge region of the porous material.

The basic idea underlying the present invention is providing even more reliable means against delamination of porous coatings from metallic neural interface devices, in order to further increase the mechanical stability of these devices. This is important for a widespread acceptance of neural interfaces for long term and human applications. As the protecting portion is in physical contact with an edge region of the porous material, the invention provides more stability and reliability for the overall neural interface device. According to the invention, physical contact of the protecting portion with the edge region of the porous material occupies at least part of the edge. This reduces physical contact of the porous material's edge with surrounding tissue. As a consequence, the forces exerted by surrounding tissue on edges of the porous material are reduced significantly. Any risk of breakage and delamination of porous material can thus be avoided in a very efficient way.

The invention even allows for a more widespread use of porous materials with flexible neural interface devices of the invention. Flexibility is often desired in order to ensure that a neural interface can adapt to uneven surfaces of neural tissues, for example to surfaces of brain structures. Flexibility can also stabilize electrical contact when the neural structure's shape changes to a certain degree over time. This variation in shape can be compensated by a neural interface that possesses sufficient flexibility and therefore follows changes in the shape of the neural structure. In the absence of the edge protection of the invention, any flexing of the flexible neural interface may intensify contact of edges of the porous material to surrounding tissue and thus increase the risk of delamination. This is avoided by the invention at every edge which is protected with a protecting portion of the securing material.

The word “neural interface device” as used herein is not limited to a specific shape. It refers to any object that can be implanted into a human tissue and that is capable of electrical interaction with adjacent neuronal tissue. Electrical interaction may include a sensing of a (physiological) electrical signal in adjacent neuronal tissue and/or a transmission of an external electrical stimulus into such tissue. A transmitting element, e.g. an electrically conductive wire, can be connected to the neural interface device, for further transmission of the (physiological) electrical signal and/or for transmitting the external electrical stimulus via the neural interface device into the tissue. The neural interface device according to the present invention is preferably a neural interface device for neurostimulation, in particular an electrode for cortical and/or deep brain stimulation. Accordingly, the expression “in electrical contact with” as used in connection with the present invention means that the contact of the porous material with the electrically conductive material is such that sensed physiological electrical signals or stimuli can be transmitted between these materials. Typically part of the porous material being in electrical contact with the electrically conductive material is in physical contact with part of the electrically conductive material.

According to the invention, the electrically conductive material can be any material which is suitable as an electrically conductive material, e.g., any material capable of transmitting the external electrical stimulus towards the neuronal tissue and/or capable of transmitting the (physiological) electrical signal towards a transmitting element that may be connected to the neural interface device. A preferred electrically conductive material comprises gold, iridium, platinum, titanium, silver, and/or palladium. A particularly preferred electrically conductive material comprises platinum Pt, platinum-iridium (Pt/lr), iridium oxide (IrOx) or titanium nitride (TiN).

The electrically conductive material can be an electrically conductive sheet material, e.g., an electrically conductive sheet material comprising ribbon-shaped sections. The porous material is preferably arranged on part of a surface of the electrically conductive sheet material, e.g., on at least one surface of at least on ribbon-shaped section.

The neural interface device of the invention comprises a porous material. Pore size of the porous materials is not limited to a specific range. The porous material can comprise micropores, mesopores and/or macropores. According to IUPAC, pores with widths not exceeding about 2 nm (20 Å) are called micropores; those with widths from 2 to 50 nm are called mesopores, and those with widths exceeding about 0.05 μm or 50 nm (500 Å) are called macropores.

A preferred porous material comprises a carbon material. Typical carbon materials are porous. This is due to the fact that carbonization of organic matter is a pyrolytic process. Gases originating from thermal decomposition of the organic matter leave an organic precursor material via channels which are pores. If, on the other hand, the carbon material is composed from already carbonized particles (e.g. from graphene, graphene oxide (GO) or reduced graphene oxide (rGO) particles or essentially planar macromolecules), there are always spaces between the particles or macromolecules. These spaces are pores which are within the carbon material due to the random orientation of the particles or macromolecules.

The carbon material is not limited to a specific carbon material. Any carbon material that can be secured by the securing material as described herein and that has suitable properties, i.e. with regard to electrical conductivity, flexibility, mechanical robustness, and inertness when exposed to neuronal tissue, is suitable in the context of the invention.

A preferred carbon material is a graphene material. Graphene materials include graphene, GO, rGO and other kinds of modified graphene. This makes it possible that the carbon material is present in the form of extremely thin carbon material layers.

Graphene is an allotrope of carbon that is consisting of single layers of atoms arranged in a two-dimensional honeycomb lattice. In practice, this ideal form of graphene is not reached as separation into single layers is not complete and functional groups, in particular oxygen functional groups are present at least at the edges of the single layers.

GO can be obtained by treating graphite with strong oxidizers. GO includes oxygen functional groups also at carbon atoms within the single layer. From M. Chhowalla et al., “Chemically Derived Graphen Oxide: Towards Large-Area Thin-Film Electronics and Optoelectronics”, Advanced Materials, 2010, Vol. 22, page 2393 to 2415, it is known that chemically derived graphene possesses a unique set of properties arising from oxygen functional groups that are introduced during chemical exfoliation of graphite.

Further, rGO can be obtained from GO by partial reduction which reduces the GO's original content of oxygen functional groups.

Most preferably, the carbon material is a GO or a rGO.

The neural interface device according to the invention comprises a securing material for securing the porous material on the electrically conductive material. The securing material may be made of any material that is suitable for securing the porous material on the electrically conductive material and that is tolerated by the surrounding tissue after implantation of the electronic device. A skilled person knows suitable securing materials.

The securing material preferably comprises a polymer, e.g., a polymer selected from polyimide, parylene, polydimethylsiloxane, and SU8. A particularly preferred polymer is a polyimide. Every physiologically acceptable polymer can be used.

The shape of the securing material is not limited. A preferred securing material is a securing layer.

According to the invention, the securing material comprises a protecting portion which is in physical contact with an edge region of the porous material.

The edge region can be a side surface of the porous material. The side surface of the porous material extends from a distal surface of the porous material to a proximal surface of the porous material. The proximal surface of the porous material is oriented towards the electrically conductive material. The distal surface of the porous material is opposite to the proximal surface of the porous material and facing away from the electrically conductive material.

Preferably, the protecting portion protrudes further from the electrically conductive material than the edge region. The protrusion of the protecting portion and of the edge region is measured orthogonal to the surface of the electrically conductive material. Such protruding protection portion is advantageous in particular when the transition from the distal surface of the porous material to the side surface of the porous material is not round, but for example, angular or rectangular. In such angular transitions of porous brittle materials, cracking can occur more easily than within planar surfaces. It turned out that a (further) protruding protection portion of the securing material protects very efficiently even against such kind of cracking.

Most preferably, the protecting portion comprises a covering portion which covers the edge region. The covering portion which covers the edge region extends on the distal surface of the porous material. The covering portion typically covers only a part of the distal surface which is adjacent to the side surface of the porous material. The additional covering portion provides additional protection to the edge region and is even more efficient against cracking at the angular transition of the porous material. It therefore provides a surplus of safety for the use of the neural interface device in humans.

Typically at least part of the porous material is arranged between different securing material areas. The securing material areas area typically part of the same securing material. However, different securing material areas can also be part of different securing materials arranged on different areas of the electrically conductive material.

The securing material can at each of the different securing material areas comprise covering portions covering opposite edge regions of the porous material. Such covering portions help to secure the edge regions in a balanced way. It is possible that the covering portions are secured at all sides, i.e. completely along the edge(s).

In a particularly preferred neural interface device according to the invention, the securing material surrounds the porous material on the electrically conductive material. This provides best mechanical protection against electrode delamination from any direction. Most preferably, the protecting portion protrudes further from the electrically conductive material than the edge region all around the edge region.

It is further preferred when the edge region of the porous material is covered all around by the covering portion. So, all portions of the edge are secured and covered.

The invention does not exclude further materials or layers. In particular, at least one additional layer may be present. The additional layer may be present, for example, between the electrically conductive material and the securing material and/or between the electrically conductive material and the porous material.

The invention further relates to a method for preparing a neural interface device according to the invention, comprising the following steps:
a) a precursor comprising an electrically conductive material, a porous material in electrical contact with the electrically conductive material, and a covering material, e.g. a covering layer, is provided, wherein the covering material comprises a protecting portion which is in physical contact with an edge region of the porous material;
b) part of the covering material is removed in an area of the covering material which covers the porous material.

Removal of a part of the covering material is typically carried out such that an active surface of the porous material is exposed. When implanted in a neural tissue, the exposed active surface will be available for electric interaction with the tissue.

A typical precursor differs from a neural interface device according to the invention in that the covering material (fully) covers the porous material. At least part of the porous material, i.e. at least part of the distal surface of the porous material, is freed on its top from the covering material in step b).

The method can include at least part of the following sequence of additional steps for forming the precursor:
Providing an electrically conductive material, e.g. by depositing an electrically conductive material on at least part of a base material,
applying a porous material, e.g., a carbon material, on at least part of the electrically conductive material,
applying a covering material, e.g., a covering layer, such that the applied covering material comprises a protecting portion which is in physical contact with an edge region of a porous material. Applying the covering material can comprise application of a covering material, e.g. a covering layer, on the entire surface of the electrically conductive material including those areas of the electrically conductive material already covered by the porous material.

These additional steps result in a precursor as defined in step a).

Preferably, step b) is carried out without removing a covering portion of the covering material which covers an edge region of the porous material. This results in a preferred neural interface device according to the invention, wherein the protecting portion comprises a covering portion which covers the edge region.

The best technique for removing part of the covering material in step b) depends on the covering material. Step b) may comprises reactive ion etching (RIE). This technique can be applied, for example, when the covering material comprises a polymer, e.g., a polyimide, or is a covering polymer layer, such as a covering polyimide layer.

It is immediately apparent that the material of the covering and securing materials, as mentioned herein, is usually the same. The securing material can be considered as a part of the covering material that is left after step b).

The invention further relates to the use of a polymer, e.g., a polymer selected from polyimide, parylene, polydimethylsiloxane, and SU8, preferably polyimide, for protecting an edge region of a porous material.

The porous material is typically in electrical contact with an electrically conductive material.

Any feature described in connection with one specific embodiment of the invention, i.e. with the neural interface device, the method or the use, also holds true for the other embodiments or is also applicable to the other embodiments, if not explicitly stated otherwise.

The invention is described in more detail by the following non-limiting figures and examples.

### It is Shown in

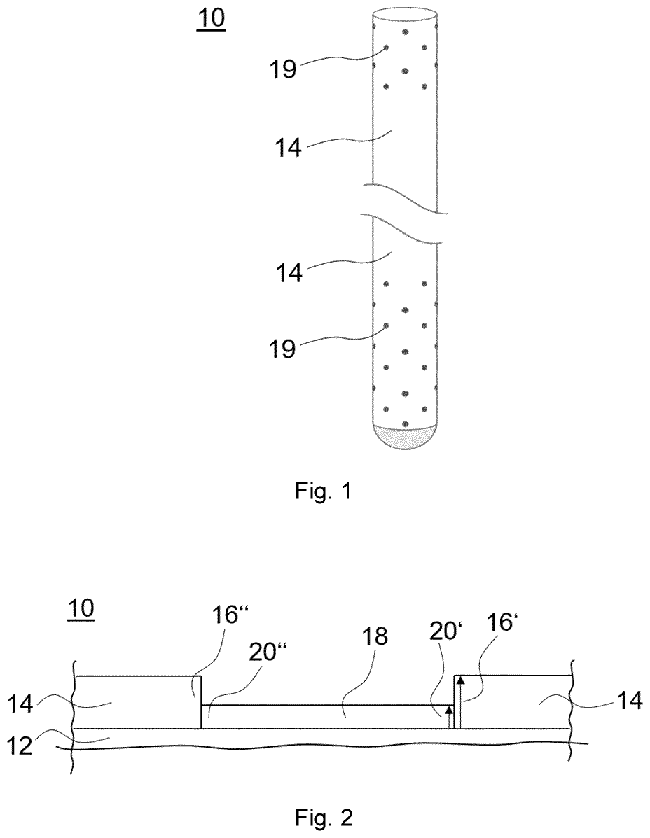

FIG. 1 an illustration of an neural interface device according to the invention;

FIG. 2 a section of a part of a neural interface device according to the invention;

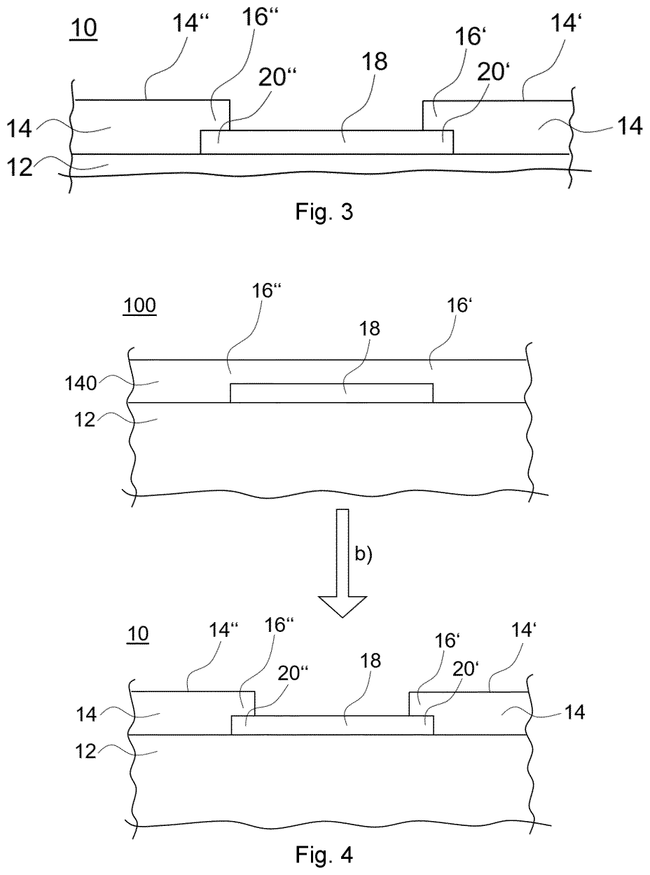

FIG. 3 a section of a part of the device shown in FIG. 1; and in

FIG. 4 an illustration of a method according to the invention for preparing the device shown in FIGS. 1 and 3; and

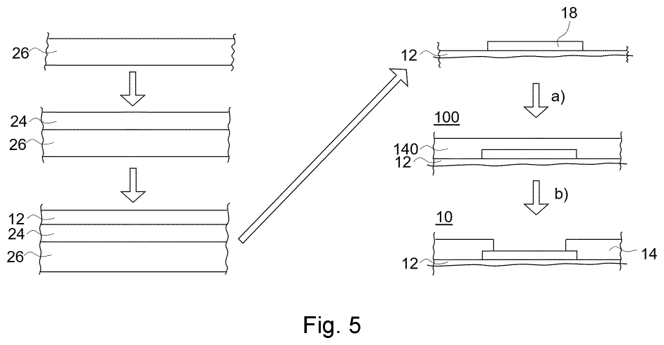

FIG. 5 a scheme illustrating a method for preparing a neural interface according to the invention.

FIG. 1 shows a neural interface device 10 according to the invention, which can be used for detecting, receiving and/or inducing physiological electrical signals, when implanted into a neural tissue.

The neural interface device 10 comprises a plurality of active areas 19 comprising porous material which is secured by securing material 14 as shown in FIG. 3.

In this specific example, the porous material is a graphene oxide material.

A securing material 14, which is polyimide, surrounds the porous material on an electrically conductive material.

In FIG. 1, the electrically conductive material is entirely covered by the porous material and the securing material.

The lowest part of FIG. 1 shows an end of the neural interface device with a group of active areas 19.

Another group of active areas 19 is shown in the upper part of FIG. 1.

FIG. 2 shows a section of a single active area of a neural interface device according to the invention.

The neural interface device 10 comprises an electrically conductive material 12, a porous material 18 in electrical contact with the electrically conductive material 12, and a securing material 14 for securing the porous material 18 on the electrically conductive material 12.

The securing material 14 comprises a protecting portion 16′ which is in physical contact with an edge region 20′ of the porous material.

Two arrows are added in this figure in order to show that protecting portion 16′ protrudes further from the electrically conductive material 12 than the edge region 20′.

The securing material 14 surrounds the porous material on the electrically conductive material 12.

FIG. 3 shows a section of a single active area 19 of a neural interface device according to FIG. 1.

This neural interface device differs from the one shown in FIG. 2 in that the protecting portion 16′ comprises a covering portion which covers the edge region 20′.

At least part of the porous material is arranged between different securing material areas 14′, 14″.

The securing material 14 (which is a polyimide layer) comprises covering portions covering opposite edge regions 20′, 20″ of the porous material 18 at each of the different securing material areas 14′, 14″.

In this specific example, the edge surrounds the circular porous material such that opposite edge regions 20′ and 20″ merge into one another and are part of one circular edge region.

Similarly, the covering portions merge into one another such that the circular edge region of the porous material 18 is covered all around by the covering portion.

FIG. 4 illustrates a method for preparing the neural interface device as shown in FIGS. 1 and 3.

A precursor 100 comprising the electrically conductive material 12, the porous material 18 (i.e. the graphene oxide material) in electrical contact with the electrically conductive material 12, and a covering material 140 made of polyimide is provided.

The covering material 140 comprises a protecting portion 16′ which is in physical contact with an edge region 20′ of the porous material.

According to the method, part of the covering material 140 is removed in an area of the covering material 140 which covers the porous material 18.

As illustrated in the lower part of FIG. 4. As can be seen in the lower part of FIG. 4, step b) is carried out without removing a covering portion of the covering material which covers an edge region 20′ of the porous material 18.

Step b) comprises reactive ion etching (RIE).

In the following, a possible embodiment of the preparation of a neural interface 10 according to the present invention shall be given:

The preparation of a neural interface 10 of the invention is described with reference to FIG. 5. It was prepared according to the following steps.

### Polyimide Deposition on a SiO₂/Si Wafer (Upper Step in FIG. 5 on the Left):

The step of depositing base material 24 (polyimide) on a SiO₂/Si wafer 26 was carried out as described by VECHERKINA, E. L. et al., Metallized polyimide films: Metallization and mechanism of the process, Polymer Science Series A 2007, Vol. 49, No. 2, pages 142 to 147.

### Deposition of Metallic Tracks:

The subsequent step of depositing an electrically conductive material 12 in the form of metallic tracks 12 on the polyimide (lower step in FIG. 5 on the left) was also carried out as described by VECHERKINA, E. L. et al., Metallized polyimide films: Metallization and mechanism of the process, Polymer Science Series A 2007, Vol. 49, No. 2, pages 142 to 147.

Coating of the Metallic Tracks with a Porous Material:

The coating of electrically conductive material 12 in the form of the metallic tracks on the base material 24 was carried out as set forth the below:

A GO suspension in water was filtered through a porous membrane thereby forming a GO structure on the membrane top. Suitable GO suspensions contain from 0.001 to 5 mg of GO per milliliter and the volume of suspension can be chosen in a range from 5 to 1000 mL.

The GO structure was transferred from the membrane onto a sacrificial substrate, whereby the GO structure was placed between the membrane at the top and the sacrificial substrate at the bottom.

The membrane was removed from the top, whereby the GO structure remained attached onto the sacrificial substrate, resulting in a GO film on sacrificial substrate which was then transferred to electrically conductive material 12 the metallic tracks on the polyimide substrate by wet transfer.

### Drying on the Hotplate at 50° C. for 10 Minutes.

The coating of the metallic tracks on the polyimide with a porous material can alternatively be carried out by other well-established transfer techniques. Such techniques have already been described and reviewed for a broad range of substrates including polyimide, and porous materials, including carbon materials (e.g. by Kang J. et al., Graphene transfer: key for applications, Nanoscale, 2012, Vol. 4, No. 18, pages 5527 to 5537).

### Patterning of the Coating in the Future Electrode Regions:

The patterning of the coating in the future electrode regions can be carried out by the techniques described and referred to by Hong J.-Y. et al. Micropatterning of graphene sheets: recent advances in techniques and applications, J. Mater. Chem., 2012, Vol. 22, pages 8179 to 8191. The patterning resulted in porous material 18 covering only the desired parts of the electrically conductive material 12 as shown at the top of FIG. 5, on the right. The sections shown in FIG. 5 on the right do not show all layers. Only the uppermost layers are shown. Wafer 26 and base material 24 are omitted.

### Formation of a Securing Material by a Second Polyimide Deposition:

The second polyimide deposition (FIG. 5, upper step a) on the right) was carried out as described above for the first polyimide deposition. This resulted in a covering material 140 comprising a protecting portion which is in physical contact with an edge region of the porous material 18.

### Holes Opening Across the Polyimide:

Parts of the covering material 140 were removed in areas of the covering material which cover porous material 18, without removing those covering portions of the covering material which cover edge regions of porous material. The removal of the parts of the covering material was achieved by reactive ion etching.

### REFERENCE SIGNS

10 neural interface device
12 electrically conductive material
14 securing material
14′, 14″ securing material area
16′, 16″ protecting portion
18 porous material
19 active area
20′, 20″ edge region
24 base material
26 wafer
100 precursor
140 covering material

1. A neural interface device comprising an electrically conductive material for transmitting electrical signals, a porous material in electrical contact with the electrically conductive material, and a securing material for securing the porous material on the electrically conductive material, wherein the securing material comprises a protecting portion which is in physical contact with an edge region of the porous material.

2. The neural interface device according to claim 1,
wherein
the porous material comprises a carbon material.

3. The neural interface device according to claim 2,
wherein
the carbon material is a graphene material.

4. The neural interface device according to claim 1,
wherein
the securing material comprises a polymer selected from polyimide, parylene, polydimethylsiloxane, and SU8.

5. The neural interface device according to claim 1,
wherein
the protecting portion protrudes further from the electrically conductive material than the edge region.

6. The neural interface device according to claim 1,
wherein the protecting portion comprises a covering portion which covers the edge region.

7. The neural interface device according to claim 1,
wherein
at least part of the porous material is arranged between different securing material areas.

8. The neural interface device according to claim 7,
wherein
the securing material at each of the different securing material areas comprises covering portions covering opposite edge regions of the porous material.

9. The neural interface device according to claim 1,
wherein
the securing material surrounds the porous material on the electrically conductive material.

10. The neural interface device according to claim 9,
wherein
the edge region of the porous material is covered all around by the covering portion.

11. A method for preparing a neural interface device, comprising the following steps:
a) a precursor comprising an electrically conductive material, a porous material in electrical contact with the electrically conductive material, and a covering material is provided, wherein the covering material comprises a protecting portion which is in physical contact with an edge region of the porous material;
b) part of the covering material is removed in an area of the covering material which covers the porous material.

12. The method according to claim 11,
wherein
step b) is carried out without removing a covering portion of the covering material which covers an edge region of the porous material.

13. The method according to claim 11,
wherein
step b) comprises reactive ion etching (RIE).

14. Method for a polymer selected from polyimide, parylene, polydimethylsiloxane, and SU8, including protecting an edge region of a porous material.

15. Method according to claim 14,
wherein
the porous material is in electrical contact with an electrically conductive material.
